(19) United States
(12) Patent Application Publication
(10) Pub. No.: US 2018/0104115 A1
(43) Pub. Date: Apr. 19, 2018

(54) Incontinence Sensor Contact System

(71) Applicants: Daniel R. Collette, Albuquerque, NM (US); Mathew P. Napier, Albuquerque, NM (US)

(72) Inventors: Daniel R. Collette, Albuquerque, NM (US); Mathew P. Napier, Albuquerque, NM (US)

(21) Appl. No.: 15/297,074

(22) Filed: Oct. 18, 2016

(51) Int. Cl. A61F 13/42; G01N 27/04

(52) U.S. Cl. CPC A61F 13/42; A61F 2013/424; G01N 27/048

(57) ABSTRACT

A sensor contact system is disclosed. In accordance with one embodiment, the sensor contact system may include more than one contact point within a single conductive element, where each contact point is separate and distinct from one or more other points in the same conductive element, thus allowing multiple, distinct, measurements to be made within the same conductive element.

### CROSS REFERENCE TO RELATED APPLICATIONS

This application claims benefit of priority from U.S. Provisional Patent Application Ser. No. 62/243,067, entitled “Incontinence Sensor Contact System” filed Oct. 18, 2015, the entire disclosure of which is incorporated herein by reference for all purposes. It is also a follow-on patent of U.S. Pat. No. 8,421,636 B2 “Patient Monitoring System” which claims incontinence products, manufactured in high speed manufacturing lines, with embedded conductive elements.

### BACKGROUND OF THE INVENTION

With an ageing population, there is a significant increase in the percentage of the population which are entering assisted living, facilities, nursing homes, Alzheimer facilities and in home care. Associated with this increase in ageing population is the demand for both facilities and caregivers. This has brought about a demand for improved services and care in an industry which has been lagging behind hospitals and clinics in funding and training. This patent is a follow on patent to one issued for a “Patient Monitoring System” U.S. Pat. No. 8,421,636 B2, Apr. 16, 2013, for use in assisted living centers, hospitals, nursing home and other facilities for the aged.

Associated with the mentioned patent, is a sensing diaper or brief which automatically notifies caregivers of a wet event so that proper care can be rendered in a timely manner. One of the greatest issues facing the incontinent elderly are complications of skin breakdown and infections caused by prolonged exposure to urine and urea byproducts. This patent introduces a novel approach to attaching the sensor to the incontinence product and algorithms to verify proper attachment, alignment and verifies that a change has occurred.

Previous embodiments of the sensing incontinence products have used snaps or pins as the mechanism of connecting to the detector. With the expanding use of adult incontinence products with sense technology, the continued use of snaps as the connecting medium increases the metallic content in landfills and introduces cost and complexity in a high-speed incontinence manufacturing lines. This provisional patent introduces a system for attachment of a detector to a sensing incontinence product without the use of snaps or other adders to the manufacturing line.

The attachment mechanism must be easily attached, secure, repeatable in its performance. An added plus in this patent are mechanisms and algorithms which ensure that the incontinence product was changed, is correctly aligned with the sensors and a verification of readiness for a wet sense event.

One of the novel concepts in this patent is the placement of a minimum of 2 contacts in each conductive sensor element strip. By contacting the conductive sensor element strip with 2 or more individual, distinct, independent contacts, as shown in “Sensor Drawing” FIG. 1, and depicted by contact labeled as A and B in conductive sensor element 1 and as C and D in conductive sensor element 2, allows a check of conductivity between contacts A and B and/or C and D. This check ensures that the alignment is correct upon re-attachment of the sensor, and guarantees that the sensor will correctly detect a wet event. Multiple contacts also increase the probability of maintaining continuous contact with movement. The details of this are discussed in “In System Incontinent Connect Detection.” Wetness sense measurements being made between conductive sensor element lines 1 and 2 are independent of measurements being made by multiple contacts A and B or C and D in the individual conductive sensor elements.

Having multiple and independently controlled contact points A, B, C and D in the conductive sensor elements allows A and B to verify contact with conductive sensor element 1 and C and D to verify contact with conductive sensor element 2.

Verification of contact with conductive sensor element 1 and conductive sensor element 2 also verifies correct alignment between the two conductive sensor elements and the connecting wetness sensor electronics. This results in the ability for the wetness sensing system to correctly detect a wet incontinent event.

Mechanisms of Connection

There is an innumerable number of ways that the sensor connectors can be attached. One embodiment would be to directly embed the contacts in the sensor PCB which gives direct contact to the incontinence sensors with the sensor module. This embodiment is shown in FIG. 6. Other embodiments would be to embed the sensor connectors in the case of the sensor module or clips or other attachment mechanisms connected by other electrical or wireless means. Connection could also be accomplished by any means which pierces the non-woven material which covers the sensor strips as a result of the manufacturing process. It is possible as well, to change the actual incontinent manufacturing process to open a hole in the fabric covering the sensor strips and would then facilitate a number of connection possibilities which don't require the ability to pierce the covering over the conductive element in the incontinence product.

### SUMMARY OF THE INVENTION

The sensor contact system described in this patent, is a new and novel approach to several of the key parameters necessary in proper and effective sensing in incontinent products. The ability to be able to ensure proper sensing attachment to any incontinent product is key. The importance of not missing an event is critical and therefore the assurance that the overall system is intact and ready to make a measurement is vital. It is equally important to know if the sensor has been detached or rendered inactive. All of these critical events have been clearly delineated along with their respective algorithms in an elegant but simple novel approach of multiple, independent, sense capability within a single conductive element.

### DETAILED DESCRIPTION OF THE PREFERRED EMBODIMENTS

In System Incontinent Connect Detection

The objective of the “In System” automated sensor connect detection algorithm is:

Sense and report if the Contact Sensor Diaper+ and Contact Sensor Diaper− are connected to the diaper sensor strips without having to introduce any external support. See FIG. 2.

Maintain maximum redundancy/reliability for the wetness sensing application.

Utilize the sensor connection element for both the wetness sensing algorithm and diaper connection algorithm See FIG. 3.

Maximize connection probability with consolidated sense nodes for wetness sensing and independent sense nodes for the diaper connection configuration. A table of definitions for symbol references used in the text and Figures is found in Table 1.

The Wet Sense Algorithm is configured with the I/O in the following conditions:

D+ Con1: Tri State

D+ Con2: Tri State

D− Con1: Output Low

D− Con2: Output Low

Rc3 Pull Up: Disabled

Wet1: Output High or Tri State depending on Sense cycle

Wet2: Output High or Tri State depending on Sense cycle.

Prior to a diaper wet event the resistance between Diaper+ and Diaper− is very large. The Diaper+ voltage is high keeping the comparator driving a “1”.

When Diaper is wet the Diaper+ and Diaper− resistance will momentarily drop and pull down the Diaper+ node lower than the comparator sense/bias voltage resulting in a wet event at the output of the comparator

A persistence check is performed on the wet event signal to determine if real wet event had occurred filtering out spurious events such as electric static discharge.

When the unit is not sensing a wet event it will be placed in an ultralow power mode to conserve battery life. Any event will immediately and autonomously wake up the unit allowing it to do the wetness check.

Details on the algorithm and the system integration of the Wet Sense Algorithm is captured in previous filing of U.S. Pat. No. 8,421,636 B2 “Patient Monitoring System”. It includes the notification methodology; visual, audio, movement, wireless transmission.

The D+ and D− Algorithms are for verifying that the wet sense circuitry is connected correctly to the incontinence product and ready to sense.

Setup conditions for the D+ Algorithm are shown below. See FIG. 5.

Wet1: Tri State

Wet2: Tri State

D+ Con1: Output High

D+ Con2: Output Low

The Algorithm is to detect if Diaper+ A and Diaper+ B are shorted together and thus to verify that the sensor connection mechanism is properly attached to the conductive element (incontinent sensor)of the incontinent product.

If Diaper+ A is shorted with Diaper+ B, by the Diaper conductive element, then Rc1 will be in parallel with a short and the Diaper+ B node will be pulled high and the D+ Connect signal will be high indicating that the D+ connection is good.

If Diaper+ A is NOT shorted with Diaper+ B, indicating a bad or no connection to the Diaper Sensor Strip, then the Diaper+ B node will be pulled down to «Vdd. This value is much less than the comparator minus input so comparator D+ connect signal will remain low indicating a good connection has NOT been made.

The setup conditions for the D− Algorithm are shown below.

Wet1: Tri State

Wet2: Tri State

D− Con1: Output Low

D− Con2: Input with a pull up resistor Rc3 enabled

The D− Algorithm is to detect if Diaper− C and Diaper− D are shorted together by the diaper sensor strip.

If Diaper− C is shorted with Diaper− D the Diaper− D node will read a zero/low, indicating the D− sensor strip is connected correctly

If Diaper− C is NOT shorted with Diaper− D then the Diaper− D node will be pulled up to Vdd and the Diaper− D input will read a one/high. Indicating the D− sensor strip is NOT connected correctly.

The System Integration of Diaper Connection Algorithm (Diaper Change)

The caregiver will attach the wetness sensing unit to a diaper.

The Diaper connection algorithm will be initiated by a button push, magnetic proximity sensor swipe or predetermined time based interval check.

If the diaper is connected the wetness sense unit will provide a predetermined response, visual (LED), audio (buzzer), movement (vibration) or wireless message (Zigbee, Bluetooth, WiFi, etc . . . ) sent to the caregiver changing the diaper that it is connected correctly.

If the diaper is not connected correctly the wetness sense unit will provide a predetermined response for one of the three cases

D+ is not connected

D− is not connect

D+ and D− are not connected

Feedback can be visual, audio, movement and or wireless message transmission. After this diaper connection algorithm has completed, the unit will immediately and autonomously switch back to the wetness sensing algorithm configuration

The System Integration of Diaper Connection Algorithm for sensor removal monitoring.

In the event the patient removes the wetness sense unit from the diaper the caregiver will be notified.

The Diaper connection algorithm will be initiated by a time based periodic event that can range from seconds to minutes depending on the users monitoring requirements.

If the diaper is connected the wetness unit will immediately switch back to wetness sense mode.

If the diaper is not connected correctly the wetness sense unit will provide a predetermined response for one of the three cases

D+ is not connected

D− is not connect

D+ and D− are not connected

A message will be sent to the care giver that the patients wetness unit has been removed or degraded in contact and requires attention.

### BRIEF DESCRIPTION OF THE DRAWINGS

FIG. 1 Incontinence product demonstrating the utilization of multiple and independent contact points into two conductive sensor elements.

FIG. 2 Example of a dedicate Wet sense circuit that does not utilize independent contact points. Multiple attachment points to increase attachment probability but does not have the ability to do an in-system and autonomous check of the attachment. Note how pins D+ A&B and D− C&D are paired together and not independent.

FIG. 3 Example of a combined incontinence connection detection circuit and a West sense circuit. Note how pins D+ and D− pins A, B, C & D are independently controlled. Also how all passive elements are connected and controlled by I/O of the controller (FPGA or micro controller).

FIG. 4 Example of how the combined circuit shown in FIG. 4. can be configured via the controller to sense Wet events. Note how independent sensing pins A, B, C and D, are paired A&B and C&D for the single task of sensing wetness greatly increasing the probability of wetness detection. The passives have also been configured to support this in-system measurement.

FIG. 5 Example of how the combined circuit shown in FIG. 5 can be configured via the controller to determine if the sensor is connected correctly to the two conductive sensor elements in the incontinence product. Note how pins A, B, C and D are independently controlled and sensing different parts of the conductive elements. The passives have also been configured to support this in-system measurement.

FIG. 6. Shows one embodiment of the sensor contact arrays, in a clamshell configuration, where alternating pins in the array, contacting the conductive element, are distinctly separate elements, and are not ganged together. This provides multiple measurements to be made within the conductive element.

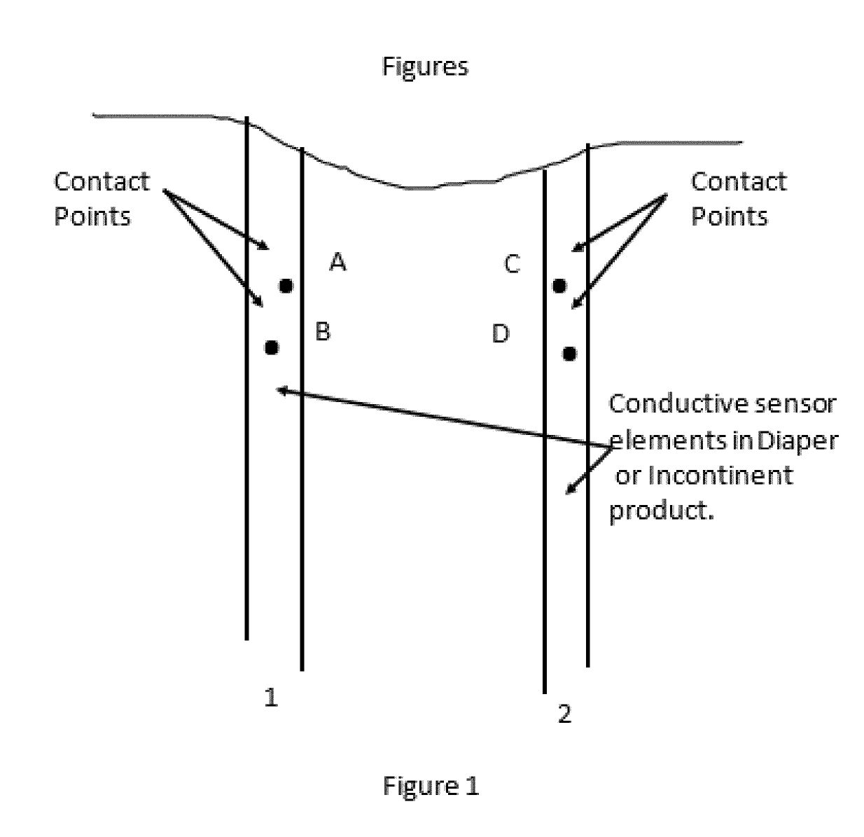

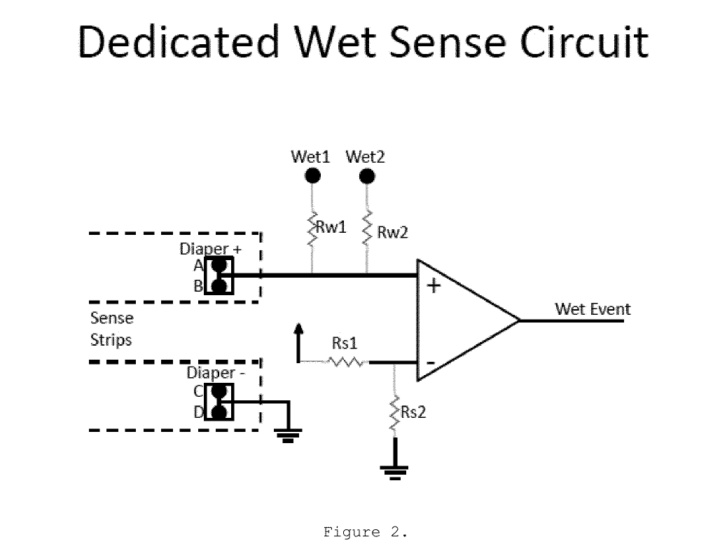

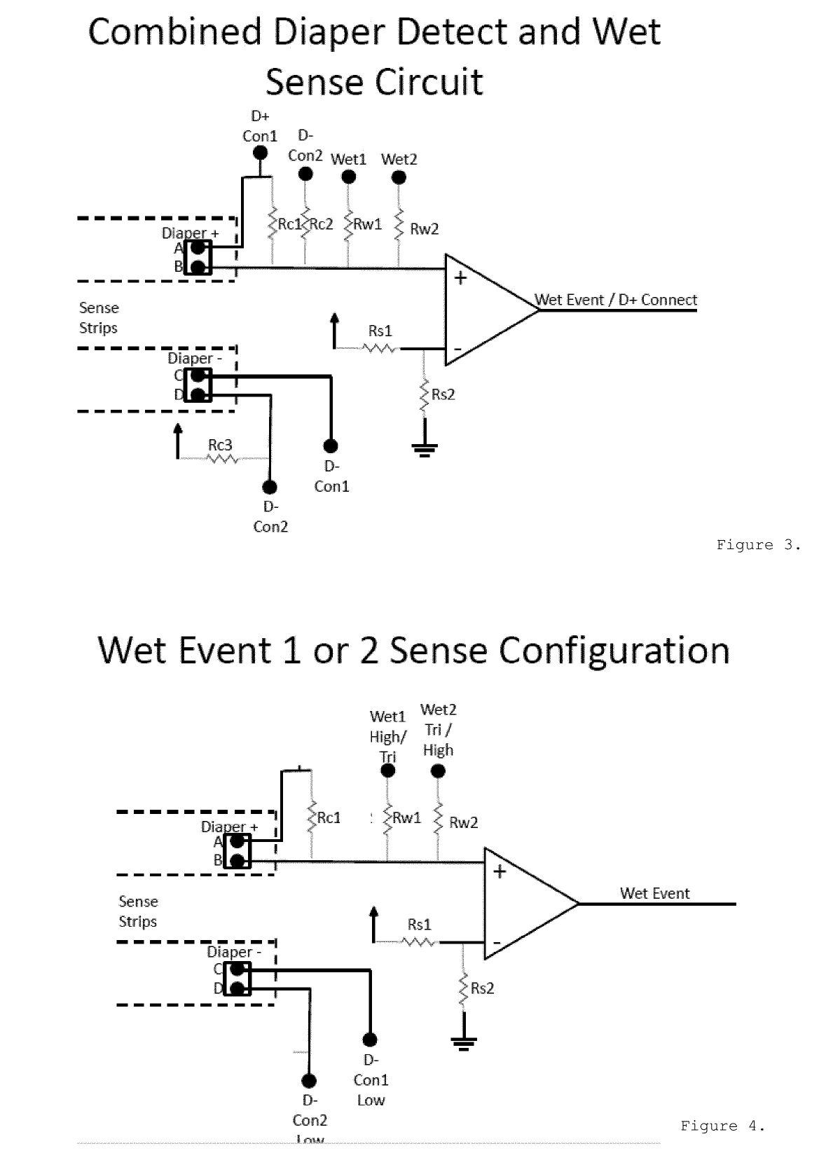

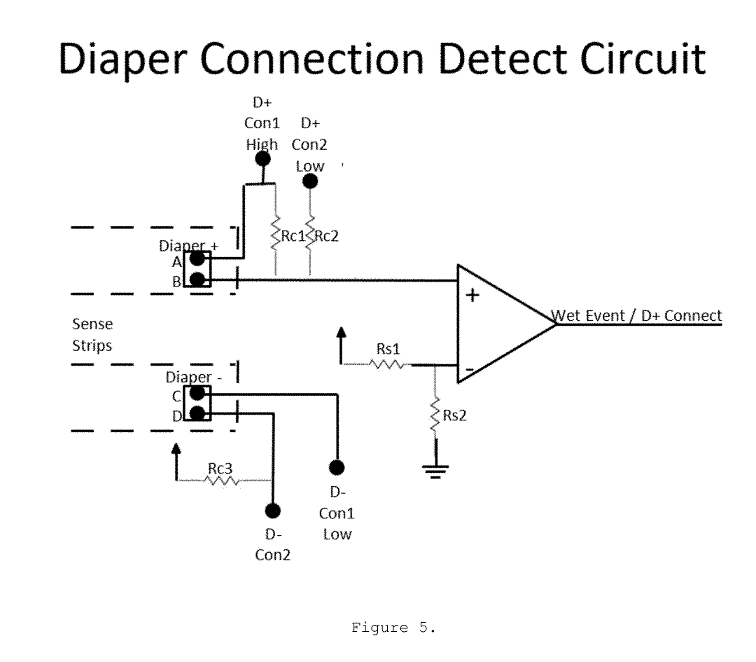

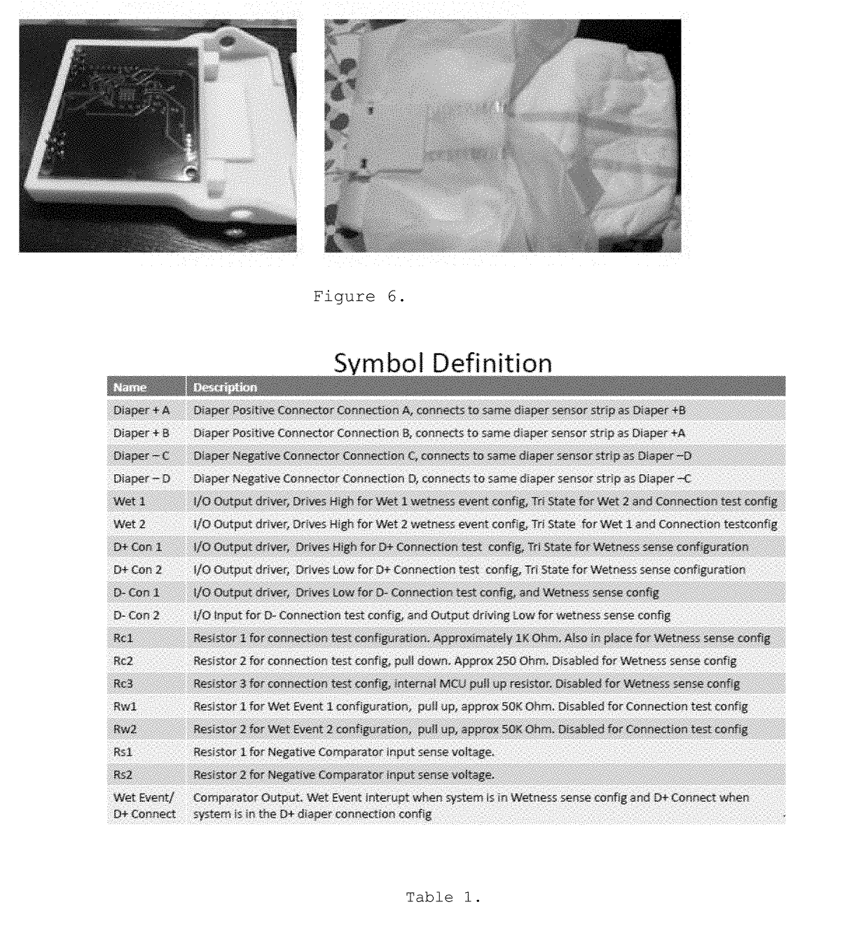

Table 1 Definition of all the symbols utilized in FIGS. 2 through 5.

What is claimed:

1. A sensor contact system comprising: two or more contact points occurring within the same conductive element, contact points are each independent from the other contact points, allowing measurements to be made between the various contact points within the same conductive element.

2. A sensor contact system according to claim 1, said sensor contact system further comprising: a variety of different conductive elements, such as metal, fabric, inks, ionic compounds, epoxies, glues, plastics, composites and skin.

3. A sensor contact system according to claim 1, said sensor contact system further comprising: contact points that are made of conductive elements, such as metal, fabric, inks, ionic compounds, epoxies, glues, plastics, composites and skin, where the conductive elements of the contact are connected to a controller, utilizing the real time configuration of I/O ports, such that entire circuits can be modified using processing elements such as FPGAs or Micro Controllers.

4. A sensor contact system according to claim 3, said sensor contact system further comprising and utilizing: an analog mux switch to allow dedicated sense circuit configurations to be switched in and out depending on the operation mode, while still maintaining the use of the same interconnection for standard sensing applications such as wetness checking in incontinence products.

5. A sensor contact system according to claim 3, said sensor contact system further comprising and utilizing: additional sense contacts in the same group (independent contact) in order to provide redundancy, reliability and advanced detection and sensing algorithms to be developed and interfaced to external systems to the sensing application.

6. A sensor contact system according to claim 3, said sensor contact system further comprising and utilizing: the capability for the utilizing system to autonomously facilitate connection detect, disconnect detect, trigger to check, and notification of wet for all types of incontinence products, including but not limited to pads, undergarments, pull-ups and tabbed products.

7. Sensory incontinent products which have designed openings in the outside impermeable membrane of the incontinence products which allow said sensor contact system as set forth in claim 3, the ability to directly contact conductive elements embedded in the incontinence products.
